(19) United States
(12) Patent Application Publication
(10) Pub. No.: US 2002/0034488 A1
(43) Pub. Date: Mar. 21, 2002

(54) Oxidizing composition and uses for dyeing, permanently reshaping or bleaching keratin fibres

(76) Inventors: Sylvain Kravtchenko, Asnieres (FR); Gregory Plos, Paris (FR)

Correspondence Address: Thomas L. Irving, FINNEGAN, HENDERSON, FARABOW,, GARRETT & DUNNER, L.L.P., 1300 I Street, N.W., Washington, DC, 20005-3315, US

(21) Appl. No.: 09/853,903

(22) Filed: May 14, 2001

(30) Foreign Application Priority Data
May 15, 2000 (FR) 00 06153

(51) Int. Cl. A61K 38/44; A61K 7/11

(52) U.S. Cl. 424/70.14; 424/94.4

(57) ABSTRACT

An oxidizing cosmetic composition for treating keratin fibers, such as human keratin fibers and more particularly human hair, comprising, in a support which is suitable for keratin fibers:

(a) at least one enzymatic oxidizing system comprising at least one enzyme chosen from 2-electron and 4-electron oxidoreductases and peroxidases,

(b) at least one maleic anhydride/(C₁-C₅)alkyl vinyl ether crosslinked copolymer; as well as to a process for improving the conservation of the enzymatic activity of an oxidizing composition for dyeing, permanently reshaping or bleaching keratin fibers, such as human hair.

The present invention relates to an oxidizing composition for treating keratin fibers, comprising at least one enzymatic oxidizing system comprising at least one enzyme of 2-electron or 4-electron oxidoreductase or peroxidase type and at least one maleic anhydride/(C₁-C₅)alkyl vinyl ether crosslinked copolymer, as well as to its uses for dyeing, permanently reshaping or bleaching keratin fibers, in such as human hair.

It is a known practice to dye keratin fibers, such as human hair, with dye compositions containing oxidation dye precursors, such as para-phenylenediamines, ortho-, or para-aminophenols, and heterocyclic bases, generally known as oxidation bases. The oxidation dye precursors, or oxidation bases, are colorless or weakly colored compounds which, when combined with oxidizing products, can give rise to colored compounds and dyes by a process of oxidative condensation.

It is also known that the shades obtained with these oxidation bases can be varied by combining them with couplers or coloration modifiers, the latter being chosen, for example, from aromatic meta-diamines, meta-aminophenols, meta-diphenols and certain heterocyclic compounds.

The variety of molecules used as oxidation bases and couplers allows a wide range of colors to be obtained.

The “permanent” coloration obtained by means of these oxidation dyes should moreover possess certain characteristics. Thus, it should have no toxicological drawbacks, it should allow shades to be obtained in the desired intensity and it should have good staying power with respect to external agents (light, bad weather, washing, permanent waving, perspiration or rubbing).

The dyes should also allow grey hair to be covered and, finally, they should be as unselective as possible, i.e. they should allow only the smallest possible differences in coloration along the same keratin fiber, which may indeed be differently sensitized (i.e. damaged) between its tip and its root.

The oxidation dyeing of keratin fibers is generally carried out in alkaline medium, in the presence of hydrogen peroxide as oxidizing agent. However, the use of alkaline media in the presence of hydrogen peroxide has the drawback of resulting in substantial degradation of the fibers, as well as appreciable decolorization of the keratin fibers, which is not always desirable.

The oxidation dyeing of keratin fibers can also be carried out using oxidizing systems other than hydrogen peroxide, such as enzymatic systems. Thus, it has already been proposed to dye keratin fibers, for example in patent application EP-A-0 310 675, with compositions comprising an oxidation dye precursor in combination with enzymes such as pyranose oxidase, glucose oxidase or uricase, in the presence of a donor for the said enzymes. Although these dyeing formulations are used under conditions which do not result in a degradation of the keratin fibers which is comparable to that caused by the dyeing operations carried out in the presence of hydrogen peroxide, they lead to colorations that may not entirely be satisfactory, either as regards the homogeneity of the color distributed along the fiber (“unison”) or as regards the chromaticity (luminosity), the dyeing power or the fastness with respect to the various attacking factors to which the hair may be subjected.

It is known that the most common technique for obtaining a permanent reshaping of the hair comprises, in a first stage, opening the keratin —S—S-disulphide (cysteine) bonds using a composition containing a suitable reducing agent (reduction step) followed, after having rinsed the head of hair thus treated, by reconstituting, in a second stage, the said disulphide bonds by applying to the hair, which has been placed under tension beforehand (rollers and the like), an oxidizing composition (oxidation step, also known as the fixing step) so as finally to give to the hair the desired shape. This technique thus makes it equally possible either to make the hair wavy or to straighten it or to remove its curliness. The new shape given to the hair by a chemical treatment such as above is remarkably long-lasting and, for example, may resist the action of washing with water or shampoos, as opposed to simple standard techniques for temporary reshaping, such as hairsetting.

The reducing compositions which may be used in order to carry out the first step of a permanent-waving operation generally contain, as reducing agents, sulphites, bisulphites, alkylphosphines or, thiols. Among the thiols, those commonly used are cysteine and the various derivatives thereof, cysteamine and the derivatives thereof, thiolactic acid or thioglycolic acid, the salts thereof and the esters thereof, such as glyceryl thioglycolate.

As regards the oxidizing compositions needed to carry out the fixing step, use is usually made in practice of compositions based on aqueous hydrogen peroxide, sodium bromate or persalts such as sodium perborate, which have the drawback of being liable to damage the hair.

The problem of the technique of the permanent-waving operations known to date is that their application to the hair can induce long-term adverse changes in the quality of the hair. The essential causes of these adverse changes in the quality of the hair are a reduction in its cosmetic properties, such as its sheen and its feel, and degradation of its mechanical properties, more particularly degradation of its mechanical strength due to swelling of the keratin fibers during the rinsing between the reduction step and the oxidation step, which can also be reflected by an increase in its porosity. The hair is weakened and can become brittle during subsequent treatments such as blow-drying.

The same problem of adverse changes in keratin fibers can be encountered during processes for bleaching the hair.

It is known that the permanent reshaping or bleaching of keratin fibers can also be carried out under milder conditions using oxidizing systems other than hydrogen peroxide, such as enzymatic systems. Thus, processes for the permanent reshaping or bleaching of keratin fibers have already been proposed, for example in patent application EP-A-0 310 675, with compositions comprising an enzyme such as pyranose oxidase, glucose oxidase or uricase, in the presence of a donor for the said enzyme. Although being used under conditions which do not result in degradation of the keratin fibers which is comparable to that caused by conventional permanent-waving or bleaching processes, these oxidizing formulations nevertheless can lead to results which are still insufficient, as regards the curl hold over time, as regards the compatibility of permanent-waved or bleached hair with subsequent treatments, as regards the reduction of the mechanical properties of the permanent-waved hair, in particular the reduction of the porosity of the hair, and as regards the reduction of the cosmetic properties such as the feel, or alternatively as regards the uniformity of the bleaching along the keratin fibers.

In addition, it has been found that the thickeners generally used in this type of enzymatic oxidizing composition of the prior art to localize it better at the point of application may not allow a conservation of the enzymatic activity which is sufficiently sustained over time.

One aim of the present invention is to solve at least one of the problems mentioned above and/or others.

The inventors have discovered, entirely surprisingly and unexpectedly, that by using a maleic anhydride/(C₁-C₅)alkyl vinyl ether crosslinked copolymer in an enzymatic oxidizing composition comprising at least one enzyme chosen from 2-electron and 4-electron oxidoreductases and peroxidases, it is found to be possible to obtain better conservation of the enzymatic activity over time.

The inventors have also discovered novel compositions containing at least one enzymatic oxidizing system comprising at least one enzyme chosen from 2-electron and 4-electron oxidoreductases and peroxidases, and at least one maleic anhydride/(C₁-C₅)alkyl vinyl ether crosslinked copolymer, and optionally, a donor for said at least one ezyme, which compositions can constitute, in the presence of oxidation dyes (oxidation bases and/or couplers), ready-to-use dye formulations which can lead to colorations that are more homogeneous and at least as intense and as chromatic as those of the prior art, without giving rise to any significant degradation, that are relatively unselective and that withstand the various attacking factors to which the hair may be subjected (light, bad weather, washing, permanent waving, perspiration or rubbing).

The inventors have also discovered that the use, in a process for the permanent reshaping of keratin fibers, of an oxidizing composition containing at least one enzymatic oxidizing system comprising at least one enzyme chosen from 2-electron and 4-electron oxidoreductases and peroxidases and at least one maleic anhydride/(C₁-C₅)alkyl vinyl ether crosslinked copolymer, and optionally a donor for said at least one enzyme, makes it possible to solve at least one of the technical problems mentioned above. For example, this type of oxidizing composition improves the curl hold obtained over time, substantially reduces the porosity of permanent-waved hair and improves the compatibility of permanent-waved hair with respect to subsequent treatments.

The inventors have also discovered, surprisingly, that the use, in a process for bleaching keratin fibers, of an oxidizing composition containing at least one enzymatic oxidizing system comprising at least one enzyme chosen from 2-electron and 4-electron oxidoreductases and peroxidases and at least one maleic anhydride/(C₁-C₅)alkyl vinyl ether crosslinked copolymer, and optionally, a donor for said at least one enzyme, makes it possible to solve at least one of the technical problems mentioned above, for example to improve the compatibility of bleached hair with respect to subsequent treatments. This type of oxidizing composition can give a more uniform bleaching effect on the hair and can improve the cosmetic properties, such as the feel.

A first subject of the present invention is thus a cosmetic oxidizing composition for treating keratin fibers, such as human keratin fibers and such as human hair, comprising, in a medium which is suitable for keratin fibers:

(a) at least one enzyme chosen from 2-electron and 4-electron oxidoreductases and peroxidases,

(b) at least one maleic anhydride/(C₁-C₅)alkyl vinyl ether crosslinked copolymer, and

(c) optionally, a donor for said at least one enzyme.

A subject of the invention is also a process for improving the conservation of the enzymatic activity of an oxidizing composition for dyeing, permanently reshaping or bleaching keratin fibers, such as human hair, and comprising, as oxidizing system, at least one enzyme chosen from 2-electron and 4-electron oxidoreductases and peroxidases, characterized in that an effective amount of a maleic anhydride/(C₁-C₅)alkyl vinyl ether crosslinked copolymer is included in the said oxidizing composition and optionally a donor for said at least one enzyme is included in said oxidizing composition.

One process according to the invention comprises a uricase oxidoreductase and uric acid as donor and a maleic anhydride/methyl vinyl ether crosslinked copolymer crosslinked with 1,9-decadiene.

### Enzymatic Oxidizing System

### 2-Electron and 4-electron Oxidoreductases and Peroxidases

The 2-electron oxidoreductases used in the oxidizing composition in accordance with the invention are used in the presence of a donor for the said enzymes and can be chosen, for example, from pyranose oxidases, glucose oxidases, glycerol oxidases, lactate oxidases, pyruvate oxidases, uricases, choline oxidases, sarcosine oxidases, bilirubin oxidases and amino acid oxidases.

According to the invention, the 2-electron oxidoreductases may be chosen from uricases of animal, microbiological and biotechnological origins.

By way of example, mention may be made of uricase extracted from boar liver, uricase from Arthrobacter globiformis, as well as uricase from Aspergillus flavus.

The 2-electron oxidoreductases can be used in pure crystalline form or in a form diluted in a diluent which is inert with respect to the said 2-electron oxidoreductase.

The 2-electron oxidoreductases in accordance with the invention may represent, for example,from 0.01 to 20% by weight approximately relative to the total weight of the dye, permanent-reshaping or bleaching composition, and such as from 0.1 to 5% by weight approximately relative to this weight.

The amount of enzyme may also be defined as a function of its activity.

The enzymatic activity of the 2-electron oxidoreductases in accordance with the invention may be defined by the oxidation of the donor under aerobic conditions.

One unit U corresponds to the amount of enzyme leading to the generation of one □mol of H₂O₂ per minute at a pH of 8.5 and at a temperature of 25° C. The amount of 2-electron oxidoreductase in accordance with the invention may range from 10 to 10⁸ units U approximately per 100 g of dye, permanent-reshaping or bleaching composition.

According to the invention, the term donor is understood to refer to the various substrates also necessary for the functioning of the said 2-electron oxidoreductases.

The nature of the donor (or substrate) for the said enzyme varies depending on the nature of the 2-electron oxidoreductase used. For example, as donors for the pyranose oxidases, mention may be made of D-glucose, L-sorbose and D-xylose; as a donor for the glucose oxidases, mention may be made of D-glucose; as donors for the glycerol oxidases, mention may be made of glycerol and dihydroxyacetone; as donors for the lactate oxidases, mention may be made of lactic acid and its salts; as donors for the pyruvate oxidases, mention may be made of pyruvic acid and its salts; as donors for the uricases, mention may be made of uric acid and its salts; as donors for the choline oxidases, mention may be made of choline and its addition salts with an acid, such as choline hydrochloride and betaine aldehyde; as donors for the sarcosine oxidases, mention may be made of sarcosine, N-methyl-L-leucine, N-methyl-DL-alanine and N-methyl-DL-valine; and lastly, as a donor for the bilirubin oxidases, mention may be made of bilirubin.

The donors (or substrates)used in accordance with the invention may represent, for example, from 0.01 to 20% by weight approximately relative to the total weight of the dye, permanent-reshaping or bleaching composition in accordance with the invention, and such as, as a further example, from 0.1 to 5% approximately relative to this weight.

The 4-electron oxidoreductases used in the composition in accordance with the invention can be chosen, for example, from laccases, tyrosinases, catechol oxidases and polyphenol oxidases.

According to one embodiment of the invention, the 4-electron oxidoreductases are chosen from laccases.

These laccases can be chosen, for example, from laccases of plant, animal, fungal yeasts, molds and fungi) and bacterial origins, the organisms of origin possibly being mono- or multicellular. The laccases can also be obtained by biotechnology.

Among the laccases of plant origin which can be used according to the invention, mention may be made of the laccases produced by plants which carry out chlorophyll synthesis, such as those mentioned in patent application FR-A-2 694 018, the specific laccases therein being incorporated by reference herein.

Mention may be made, for example, of the laccases present in extracts of Anacardiacea plants such as, for example, extracts of Magnifera indica, of Schinus molle or of Pleiogynium timoriense; in extracts of Podocarpacea plants; of Rosmarinus off.; of Solanum tuberosum; of Iris sp.; of Coffea sp.; of Daucus carrota; of Vinca minor; of Persea americana; of Catharanthus roseus; of Musa sp.; of Malus pumila; of Gingko biloba; of Monotropa hypopithys (Indian pipe), of Aesculus sp.; of Acer pseudoplatanus; of Prunus persica and of Pistacia palaestina.

Among the laccases of fungal origin, optionally obtained by biotechnology, which can be used according to the invention, mention may be made of the laccases obtained from Polyporus versicolor, from Rhizoctonia praticola and from Rhus vernicifera as described, for example, in patent applications FR-A-2 112 549 and EP-A-504 005, the descriptions of said laccases being specifically incorporated by reference herein; the laccases described in patent applications WO 95/07988, WO 95/33836, WO 95/33837, WO 96/00290, WO 97/19998 and WO 97/19999, the description of such laccases being specifically incorporated by reference herein, such as, for example, the laccases obtained from Scytalidium, from Polyporus pinsitus, from Myceliophthora thermophila, from Rhizoctonia solani, from Pyricularia orizae, and variants thereof. Mention may also be made of the laccases obtained from Trametes versicolor, from Fomes fomentarius, from Chaetomium thermophile, from Neurospora crassa, from Colorius versicol, from Botrytis cinerea, from Rigidoporus lignosus, from Phellinus noxius, from Pleurotus ostreatus, from Aspergillus nidulans, from Podospora anserina, from Agaricus bisporus, from Ganoderma lucidum, from Glomerella cingulata, from Lactarius piperatus, from Russula delica, from Heterobasidion annosum, from Thelephora terrestris, from Cladosporium cladosporioides, from Cerrena unicolor, from Coriolus hirsutus, from Ceriporiopsis subvermispora, from Coprinus cinereus, from Panaeolus papilionaceus, fromPanaeolus sphinctrinus, from Schizophyllum commune, from Dichomitius squalens, and from variants thereof.

Laccases of fungal origin, optionally obtained by biotechnology, may be chosen.

The enzymatic activity of the laccases used in accordance with the invention and having syringaldazine among their substrates can be defined by the oxidation of syringaldazine under aerobic conditions. One Lacu unit corresponds to the amount of enzyme which catalyses the conversion of 1 mmol of syringaldazine per minute at a pH of 5.5 and at a temperature of 30° C. One U unit corresponds to the amount of enzyme which produces an absorbance delta of 0.001 per minute at a wavelength of 530 nm, using syringaldazine as substrate, at 30° C. and at a pH of 6.5. The enzymatic activity of the laccases used according to the invention can also be defined by the oxidation of para-phenylenediamine. One ulac unit corresponds to the amount of enzyme which produces an absorbance delta of 0.001 per minute at a wavelength of 496.5 nm, using para-phenylenediamine as substrate (64 mM), at 30° C. and at a pH of 5.

In general, the 4-electron oxidoreductases in accordance with the invention may represent, for example, from 0.01% to 20% by weight approximately relative to the total weight of the dye, permanent-reshaping or bleaching composition, and such as, for further example,from 0.1% to 5% by weight approximately relative to this weight.

In one embodiment, and when at least one laccase is used, the amount of laccases present in the dye, permanent-reshaping or bleaching composition in accordance with the invention will vary as a function of the nature of the laccases used. For example, the amount of laccases ranges from 0.5 to 2000 Lacu approximately (i.e. from 10,000 to 40×10⁶ U units approximately or alternatively from 20 to 20×10⁶ ulac units) per 100 g of dye, permanent-reshaping or bleaching composition.

The peroxidases used in the dye, permanent-reshaping or bleaching composition in accordance with the invention can be chosen, for example, from enzymes belonging to the subclass 1.11.1 described in the book Enzyme Nomenclature, Academic Press Inc., 1984, the description of enzymes belonging to said subclass being specifically incorporated by reference herein. Some of these enzymes require the presence of a donor to function. This is the case, for example, for the NADH peroxidases (1.11.1) [donor=NADH], the fatty acid peroxidases (1.11.1.3) [donor=fatty acid, for example palmitate], the NADPH peroxidases (1.11.1.2) [donor=NADPH], the cytochrome-c peroxidases (1.11.1.5) [donor=ferrocytochrome c], the iodide peroxidases (1.11.1.8) [donor=iodide], the chloride peroxidases (1.11.1.10) [donor=chloride], the L-ascorbate peroxidases (1.11.1.1 1) [donor=L-ascorbate] and the glutathione peroxidases (1.11.1.9) [donor=glutathione].

Other peroxidases function without a donor; this is the case for the catalases (1.11.1.6) and the simplex peroxidases (1.11.1.7).

According to the invention, simplex peroxidases (1.11.1.7) may be used.

All the peroxidases function in the presence of hydrogen peroxide, which is provided in its native form or generated in situ via an enzymatic route [2-electron oxidase(s) and donor(s) in the presence of air].

The peroxidases used can be of plant, animal, fungal and bacterial origins. They can also be obtained by biotechnology.

Thus, the peroxidases can be obtained, for example, from apple, apricot, barley, black radish, beetroot, cabbage, carrot, corn, cotton, garlic, grape, mint, rhubarb, soybean, spinach, inky cap, cow's milk or microorganisms such as Acetobacter peroxidans, Staphylococcus faecalis or Arthromyces ramosus.

The unit of activity of simplex peroxidase (1.11.1.7) can be defined as being the amount of simplex enzyme forming 1 mg of purpurogallin from pyrogallol in 20 s at pH 6 and at 20° C. By way of example, black radish peroxidase P6782 from Sigma has an activity of about 250 units per mg.

The working concentration of this type of enzyme thus range, for example, from 25 to 5×10⁶ units per 100 g of composition.

The peroxidases in accordance with the invention may represent from 0.0001% to 20% by weight approximately relative to the total weight of the dye, permanent-reshaping or bleaching composition, and such as from 0.001% to 10% by weight approximately relative to this weight.

### Maleic anhydride/(C₁-C₅)alkyl vinyl ether crosslinked copolymers

The gels obtained from hydrolysed and neutralized maleic anhydride/(C₁-C₅)alkyl vinyl ether crosslinked copolymers are known in cosmetic compositions for caring for or treating the hair or the skin. They are disclosed in U.S. Pat. Nos. 5,034,220, 5,032,391, 5,024,779 and 5,254,636 from the company GAF Chemicals Corporation. The polymerization process leading to the synthesis of these polymers is disclosed in U.S. Pat. No. 5,034,488 from the said company.

The maleic anhydride/(C₁-C₅)alkyl vinyl ether crosslinked copolymers can be prepared by polymerization of maleic anhydride, of an alkyl vinyl ether and of a crosslinking agent, in the presence of a free-radical initiator, in a suitable solvent.

In one embodiment, the solvent used is a co-solvent system comprising about 45 to 65% cyclohexane and about 35-55% by weight ethyl acetate.

The polymerization can be carried out at a temperature from 0° C. to 150° C., such as from 50° C. to 100° C., and further such as from 60° C. to 80° C.

The amount of crosslinking agent generally ranges from about 1 to about 5 mol relative to the monoalkyl vinyl ether.

These crosslinking agents are chosen, for example, from divinyl ethers of aliphatic diols and divinyl ethers of polyethylene glycols and also from 1,7-octadiene, 1,9-decadiene, divinylbenzene, N,N′-bis(methyleneacrylamide), polyethylene glycol diacrylate, propylene glycol diacrylate, trimethylolpropane triacrylate, and polyhydric alcohols esterified with acrylic acid. For example, according to one embodiment, 1,9-decadiene may be used.

The free-radical initiators, which may be used in proportions ranging from 0.001% to 1% by weight relative to the monomers, are chosen, for example, from azobis(isobutyronitrile), azobis(2,4-dimethylvaleronitrile) and benzoyl, lauroyl, caprylyl, acetyl, acetylbenzoyl or di-tert-butyl peroxide, or mixtures of these compounds.

The copolymers that can be obtained after filtration and drying are in the form of powder.

They can be hydrolysed and neutralized in a basic aqueous solution such as an aqueous 10% sodium hydroxide or potassium hydroxide solution, or an aqueous 30% ammonia solution, or a solution of an alkanolamine such as monoethanolamine, diethanolamine, triethanolamine, aminomethylpropanol or aminomethylpropanediol.

According to the present invention, the viscosity of an aqueous solution containing from 0.5% to 1% by weight of the said hydrolysed and neutralized copolymer, at a pH ranging from 5 to 10, and measured at 25° C. using a Brookfield RTV viscometer with a No. 7 rotor at 20 rpm, may range from 45,000 to 70,000 mpa.s

The maleic anhydride/(C₁-C₅)alkyl vinyl ether crosslinked copolymers according to one embodiment of the present invention also may have a particle size of less than 850 microns, such as less than 75 microns and, as a further example, from 1 to 15 microns.

According to one embodiment, the maleic anhydride/(C₁-C₅)alkyl vinyl ether crosslinked copolymer may be the maleic anhydride/methyl vinyl ether copolymer, crosslinked with 1,9-decadiene, for which an aqueous 0.5% by weight solution at pH 7 has a viscosity at 250° C., measured using a Brookfield RTV viscometer with a No. 7 rotor at 20 rpm, from 45 000 to 70 000 mPa.s with a particle size of less than 75 microns, and which is sold under the name Stabileze QM by the company I.S.F.

In the composition according to the present invention, the concentration of maleic anhydride/(C₁-C₅)alkyl vinyl ether crosslinked copolymer can range, for example, from

0.001% to 10% by weight approximately relative to the total weight of the composition, such as, for further example, from 0.01% to 5% approximately.

A subject of the present invention is also a ready-to-use composition for the oxidation dyeing of keratin fibers, and for example, human keratin fibers, such as the hair, of the type comprising, in a medium which is suitable for dyeing, at least one oxidation dye, and which is characterized in that it contains:

(a) at least one enzyme chosen from 2-electron and 4-electron oxidoreductases and peroxidases,

(b) at least one maleic anhydride/(C₁-C₅)alkyl vinyl ether crosslinked copolymer.

The oxidation dyes which may be used according to the invention are chosen from oxidation bases and/or couplers.

The ready-to-use dye compositions according to the invention may contain at least one oxidation base.

The nature of these oxidation bases is not critical. They may be chosen, for example, from the ortho- and para-phenylenediamines, double bases, ortho- and para-aminophenols and heterocyclic bases below, as well as the addition salts of all these compounds with an acid.

Mention may be made, for example, of:

(I) the para-phenylenediamines of formula (I) below, and the addition salts thereof with an acid:

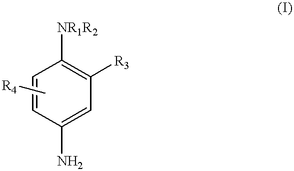

in which:

R₁ represents a hydrogen atom, a C₁-C₄ alkyl radical, a C₁-C₄ monohydroxyalkyl radical, a C₂-C₄ polyhydroxyalkyl radical, a (C₁-C₄)alkoxy(C₁-C₄)alkyl radical, a C₁-C₄ alkyl radical substituted with a nitrogenous group, a phenyl radical or a 4′-aminophenyl radical;

R₂ represents a hydrogen atom, a C₁-C₄ alkyl radical, a C₁-C₄ monohydroxyalkyl radical, a C₂-C₄ polyhydroxyalkyl radical, a (C₁-C₄)alkoxy(C₁-C₄)alkyl radical or a C₁-C₄ alkyl radical substituted with a nitrogenous group;

R₃ represents a hydrogen atom, a halogen atom such as a chlorine atom, a C₁-C₄ alkyl radical, a sulpho radical, a carboxyl radical, a C₁-C₄ monohydroxyalkyl radical, a C₁-C₄ hydroxyalkoxy radical, an acetylamino(C₁-C₄)alkoxy radical, a C₁-C₄ mesylaminoalkoxy radical or a carbamoylamino(C₁-C₄)alkoxy radical,

R₄ represents a hydrogen or halogen atom or a C₁-C₄ alkyl radical.

Among the nitrogenous groups of formula (I) above, mention may be made, for example, of amino, mono(C₁-C₄)alkylamino, di(C₁-C₄)alkylamino, tri(C₁-C₄)alkylamino, monohydroxy(C₁-C₄)alkylamino, imidazolinium and ammonium radicals.

Among the para-phenylenediamines of formula (I) above, mention may be made, for example, of para-phenylenediamine, para-toluylenediamine, 2-chloro-para-phenylenediamine, 2,3-dimethyl-para-phenylenediamine, 2,6-dimethyl-para-phenylenediamine, 2,6-diethyl-para-phenylenediamine, 2,5-dimethyl-para-phenylenediamine, N,N-dimethyl-para-phenylenediamine, N,N-diethyl-para-phenylenediamine, N,N-dipropyl-para-phenylenediamine, 4-amino-N, N-diethyl-3-methylaniline, N,N-bis(β-hydroxyethyl)-para-phenylenediamine, 4-amino-N,N-bis(β-hydroxyethyl)-2-methylaniline, 4-amino-2-chloro-N ,N-bis(β-hydroxyethyl)aniline, 2-β-hydroxyethyl-para-phenylenediamine, 2-fluoro-para-phenylenediamine, 2-isopropyl-para-phenylenediamine, N-(β-hydroxypropyl)-para-phenylenediamine, 2-hydroxymethyl-para-phenylenediamine, N,N-dimethyl-3-methyl-para-phenylenediamine, N,N-(ethyl- β-hydroxyethyl)-para-phenylenediamine, N-(βΥ-dihydroxypropyl)-para-phenylenediamine, N-(4′-aminophenyl)-para-phenylenediamine, N-phenyl-para-phenylenediamine, 2-□-hydroxy-ethyloxy-para-phenylenediamine, 2-β-acetylaminoethyloxy-para-phenylenediamine, N-(β-methoxyethyl)-para-phenylenediamine and 2-methyl-1-N-β-hydroxyethyl-para-phenylenediamine, and the addition salts thereof with an acid.

Among the para-phenylenediamines of formula (I) above, para-phenylenediamine, para-toluylenediamine, 2-isopropyl-para-phenylenediamine, 2-β-hydroxyethyl-para-phenylenediamine, 2- -hydroxyethyloxy-para-phenylenediamine, 2,6-dimethyl-para-phenylenediamine, 2,6-diethyl-para-phenylenediamine, 2,3-dimethyl-para-phenylenediamine, N,N-bis(β-hydroxyethyl)-para-phenylenediamine, 2-chloro-para-phenylenediamine and the addition salts thereof with an acid may be used.

(II) According to the invention, the term double bases is understood to refer to the compounds containing at least two aromatic rings bearing amino and/or hydroxyl groups.

Among the double bases which can be used as oxidation bases in the dye compositions in accordance with the invention, mention may be made, for example, of the compounds corresponding to formula (II) below, and the addition salts thereof with an acid:

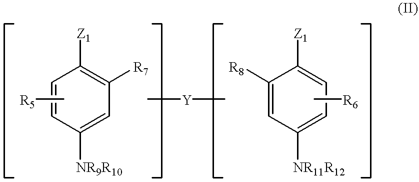

in which:

Z₁ and Z₂, which may be identical or different, represent a hydroxyl or —NH₂ radical which may be substituted with a C₁-C₄ alkyl radical or with a linker arm Y;

the linker arm Y represents a linear or branched alkylene chain containing from I to 14 carbon atoms, which may be interrupted by or terminated with one or more nitrogenous groups and/or one or more hetero atoms such as oxygen, sulphur or nitrogen atoms, and optionally substituted with one or more hydroxyl or C₁-C₄ alkoxy radicals;

R₅ and R₆, which may be identical or different, represent a hydrogen or halogen atom, a C₁-C₄ alkyl radical, a C₁-C₄ monohydroxyalkyl radical, a C₂-C₄ polyhydroxyalkyl radical, a C₁-C₄ aminoalkyl radical or a linker arm Y;

R₇, R₈, R₉, R₁₀, R₁₁ and R₁₂, which may be identical or different, represent a hydrogen atom, a linker arm Y or a C₁-C₄ alkyl radical; it being understood that the compounds of formula (II) contain only one linker arm Y per molecule.

Among the nitrogenous groups of formula (II) above, mention may be made, for example, of amino, mono(C₁-C₄)alkylamino, di(C₁-C₄)alkylamino, tri(C₁-C₄)alkylamino, monohydroxy(C₁-C₄)alkylamino, imidazolinium and ammonium radicals.

Among the double bases of formula (II) above, mention may be made, for example, of N,N′-bis(β-hydroxyethyl)-N,N′-bis(4′-aminophenyl)-1,3-diaminopropanol, N,N′-bis(β-hydroxyethyl)-N,N′-bis(4′-aminophenyl)ethylenediamine, N,N′-bis(4-aminophenyl)-tetramethylenediamine, N,N′-bis(β-hydroxyethyl)-N,N′-bis(4-aminophenyl)-tetramethylenediamine, N,N′-bis(4-methylaminophenyl)tetramethylenediamine, N,N′-bis-(ethyl)-N,N′-bis(4′-amino-3′-methylphenyl)ethylenediamine and 1 ,8-bis(2,5-diaminophenoxy)-3,5-dioxaoctane, and the addition salts thereof with an acid.

Among these double bases of formula (II), N,N′-bis(β-hydroxyethyl)-N,N′-bis(4′-aminophenyl)-1,3-diaminopropanol and 1,8-bis(2,5-diaminophenoxy)-3,5-dioxaoctane, or one of the addition salts thereof with an acid, may be used.

(III) The para-aminophenols corresponding to formula (III) below, and the addition salts thereof with an acid:

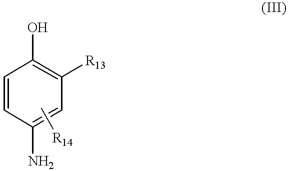

in which:

R₁₃ represents a hydrogen atom, a halogen atom such as fluorine, a C₁-C₄ alkyl, C₁-C₄ monohydroxyalkyl, (C₁-C₄)alkoxy(C₁-C₄)alkyl, C₁-C₄ aminoalkyl or hydroxy(C₁-C₄)alkylamino(C₁-C₄)alkyl radical,

R₁₄ represents a hydrogen atom, a halogen atom such as fluorine, a C₁-C₄-alkyl, C₁-C₄ monohydroxyalkyl, C₂-C₄ polyhydroxyalkyl, C₁-C₄ aminoalkyl, C₁-C₄ cyanoalkyl or (C₁-C₄)alkoxy-(C₁-C₄)alkyl radical.

Among the para-aminophenols of formula (III) above, mention may be made, for example, of para-aminophenol, 4-amino-3-methylphenol, 4-amino-3-fluorophenol, 4-amino-3-hydroxymethylphenol, 4-amino-2-methylphenol, 4-amino-2-hydroxymethylphenol, 4-amino-2-methoxymethylphenol, 4-amino-2-aminomethylphenol, 4-amino-2-(β-hydroxyethylaminomethyl)phenol, and the addition salts thereof with an acid.

(IV) The ortho-aminophenols which can be used as oxidation bases in the context of the present invention are chosen, for example, from 2-aminophenol, 2-amino-1-hydroxy-5-methylbenzene, 2-amino-1-hydroxy-6-methylbenzene and 5-acetamido-2-aminophenol, and the addition salts thereof with an acid.

(V) Among the heterocyclic bases which can be used as oxidation bases in the dye compositions in accordance with the invention, mention may be made, for example, of pyridine derivatives, pyrimidine derivatives, pyrazole derivatives, and the addition salts thereof with an acid.

Among the pyridine derivatives, mention may be made, for example, of the compounds described, for example, in patents GB 1,026,978 and GB 1,153,196, the description of the pyridine derivatives being specifically incorporated herein by reference, such as 2,5-diaminopyridine, 2-(4-methoxyphenyl)amino-3-aminopyridine, 2,3-diamino-6-methoxypyridine, 2-(β-methoxyethyl)amino-3-amino-6-methoxypyridine and 3,4-diaminopyridine, and the addition salts thereof with an acid.

Among the pyrimidine derivatives which may be mentioned, for example, of the compounds described, for example, in German patent DE 2,359,399 or Japanese patents JP 88-169,571 and JP 91-10659 or patent application WO 96/15765, the description of the pyrimidine derivatives being specifically incorporated herein by reference, such as 2,4,5,6-tetraaminopyrimidine, 4-hydroxy-2,5,6-triaminopyrimidine, 2-hydroxy-4,5,6-triaminopyrimidine, 2,4-dihydroxy-5,6-diaminopyrimidine and 2,5,6-triaminopyrimidine, and pyrazolo-pyrimidine derivatives such as those mentioned in patent application FR-A-2 750 048, the description of the pyrazolo-pyrimidine derivatives being specifically incorporated herein by reference, and among which mention may be made of pyrazolo[1,5-a]pyrimidine-3,7-diamine; 2,5-dimethylpyrazolo[1,5-a]pyrimidine-3,7-diamine; pyrazolo[1,5-a]pyrimidine-3,5-diamine; 2,7-dimethylpyrazolo[1,5-a]pyrimidine-3,5-diamine; 3-aminopyrazolo [1,5-a]pyrimidin-7-ol; 3-aminopyrazolo[1,5-a]pyrimidin-5-ol; 2-(3-aminopyrazolo[1,5-a]pyrimidin-7-ylamino)ethanol, 2-(7-aminopyrazolo[1,5-a]pyrimidin-3-ylamino)ethanol, 2-[(3-aminopyrazolo[1,5-apyrimidin-7-yl)-(2-hydroxyethyl)amino]ethanol, 2-[(7-aminopyrazolo[1,5-a]pyrimidin-3-yl)-(2-hydroxyethyl)amino]ethanol, 5,6-dimethylpyrazolo[1,5-a]pyrimidine-3,7-diamine, 2,6-dimethylpyrazolo[1,5-a]pyrimidine-3,7-diamine and 2,5,N7,N7-tetramethylpyrazolo[1,5-a]pyrimidine-3,7-diamine, 3-amino-5-methyl-7-imidazolylpropylaminopyrazolo[1,5-a]pyrimidine, and the addition salts thereof and the tautomeric forms thereof, when a tautomeric equilibrium exists, and the addition salts thereof with an acid.

Among the pyrazole derivatives which may be mentioned, for example, of the compounds described in patents DE 3,843,892 and DE 4,133,957 and patent applications WO 94/08969, WO 94/08970, FR-A-2,733,749 and DE 195 43 988, the description of the pyrazole derivatives being specifically incorporated herein by reference, such as 4,5-diamino-1-methylpyrazole, 3,4-diaminopyrazole, 4,5-diamino-1-(4′-chlorobenzyl)pyrazole, 4,5-diamino-1,3-dimethylpyrazole, 4,5-diamino-3-methyl-1-phenylpyrazole, 4,5-diamino-1-methyl-3-phenylpyrazole, 4-amino-1,3-dimethyl-5-hydrazinopyrazole, 1-benzyl-4,5-diamino-3-methylpyrazole, 4,5-diamino-3-tert-butyl-1-methylpyrazole, 4,5-diamino-1-tert-butyl-3-methylpyrazole, 4,5-diamino-1-(β-hydroxyethyl)-3-methylpyrazole, 4,5-diamino-1-ethyl-3-methylpyrazole, 4,5-diamino-1-ethyl-3-(4′-methoxyphenyl)pyrazole, 4,5-diamino-1-ethyl-3-hydroxymethylpyrazole, 4,5-diamino-3-hydroxymethyl-1-methylpyrazole, 4,5-diamino-3-hydroxymethyl-1-isopropylpyrazole, 4,5-diamino-3-methyl-1-isopropylpyrazole, 4-amino-5-(2′-aminoethyl)amino-1,3-dimethylpyrazole, 3,4,5-triaminopyrazole, 1-methyl-3,4,5-triaminopyrazole, 3,5-diamino-1-methyl-4-methylaminopyrazole and 3,5-diamino-4-(β-hydroxyethyl)amino-1-methylpyrazole, and the addition salts thereof with an acid.

According to one embodiment of the present invention, the oxidation bases may represent, for example, from 0.0005% to 12% by weight approximately relative to the total weight of the composition and, as a further example, from 0.005% to 8% by weight approximately relative to this weight.

The couplers which may be used are those used conventionally in oxidation dye compositions, i.e. meta-phenylenediamines, meta-aminophenols and meta-diphenols, mono- or polyhydroxylated naphthalene derivatives, sesamol and its derivatives and heterocyclic compounds such as, for example, indole couplers, indoline couplers and pyridine couplers, and the addition salts thereof with an acid.

These couplers can be chosen, for example, from 2-methyl-5-aminophenol, 5-N-(β-hydroxyethyl)amino-2-methylphenol, 3-aminophenol, 1,3-dihydroxybenzene, 1,3-dihydroxy-2-methylbenzene, 4-chloro-1,3-dihydroxybenzene, 2,4-diamino-1-(β-hydroxyethyloxy)benzene, 2-amino-4-(β-hydroxyethylamino)-1-methoxybenzene, 1,3-diaminobenzene, 1,3-bis(2,4-diaminophenoxy)propane, 1-amino-2-methoxy-4,5-methylenedioxybenzene, sesamol, α-naphthol, 6-hydroxyindole, 4-hydroxyindole, 4-hydroxy-N-methylindole, 6-hydroxyindoline, 3-(4-hydroxy-1-methyl-1H-indol-5-ylmethyl)-1-methylpyridinium chloride, 2,6-dihydroxy-4-methylpyridine, 1H-3-methylpyrazol-5-one and 1-phenyl-3-methylpyrazol-5-one, and the addition salts thereof with an acid.

When they are present, these couplers may represent, for example, from 0.0001 to 10% by weight approximately relative to the total weight of the ready-to-use dye composition, and as a further example, from 0.005 to 5% by weight approximately relative to this weight.

In general, the addition salts with an acid which can be used in the context of the dye compositions of the invention (oxidation bases and couplers) are chosen, for example, from the hydrochlorides, hydrobromides, sulphates, tartrates, lactates and acetates.

The dye composition of the invention can also comprise, in addition to the oxidation bases defined above and the optional combined couplers, direct dyes to enrich the shades with glints. These direct dyes can be chosen, for example, from neutral, cationic or anionic nitro dyes, azo dyes or anthraquinone dyes, in a weight proportion, for example, of approximately 0.001% to 20% and as a further example, from 0.01% to 10%, relative to the total weight of the composition.

The subject of the invention is also a process for dyeing keratin fibers, and, for example, human keratin fibers, such as the hair, using the ready-to-use dye composition as defined above.

According to this process, at least one ready-to-use dye composition as defined above is applied to the fibers, for a period which is sufficient to develop the desired coloration, after which the fibers are rinsed, optionally washed with shampoo, rinsed again and dried.

The time required to develop the coloration on the keratin fibers is generally, for example, from 3 to 60 minutes, a further example being from 5 to 40 minutes.

According to one specific embodiment of the invention, the process includes a first step which comprises separately storing, on the one hand, a composition (A) comprising, in a medium which is suitable for dyeing, at least one oxidation dye as defined above, and, on the other hand, a composition (B) comprising, in a medium which is suitable for dyeing, at least one enzymatic oxidizing system containing at least one enzyme chosen from 2-electron and 4-electron oxidoreductases and peroxidases and at least one maleic anhydride/(C₁-C₅)alkyl vinyl ether crosslinked copolymer, and then in mixing them together at the time of use, before applying this mixture to the keratin fibers.

According to another specific embodiment of the invention, the maleic anhydride/(C₁-C₅)alkyl vinyl ether crosslinked copolymer is incorporated into composition (A).

Another subject of the invention is a multi-compartment dyeing device or “kit” or any other multi-compartment packaging system, a first compartment of which comprises composition (A) as defined above and a second compartment of which comprises composition (B) as defined above. These devices can be equipped with means for applying the desired mixture to the hair, such as the devices described in patent FR-2,586,913, the description of such devices being specifically incorporated by reference herein.

A subject of the present invention is also a novel process for treating keratin fibers, such as human hair, in order to obtain a permanent reshaping of these fibers, for example in the form of permanent-waved hair, this process comprising the following steps: (i) a reducing composition is applied to the keratin fibers to be treated, the keratin fibers being placed under mechanical tension before, during or after the said application, (ii) the keratin fibers are optionally rinsed, (iii) an oxidizing composition as defined above is applied to the optionally rinsed keratin fibers, (iv) the keratin fibers are optionally rinsed again.

The first step (i) of this process comprises applying a reducing composition to the hair. This application is carried out lock by lock or all at once.

The reducing composition comprises, for example, at least one reducing agent, which can be chosen, for example, from thioglycolic acid, cysteine, cysteamine, glyceryl thioglycolate, thiolactic acid or thiolactic or thioglycolic acid salts.

The usual step for placing the hair under tension in a shape corresponding to the desired final shape for this hair (for example curls) can be carried out by any suitable means, such as mechanical means, known per se for maintaining the hair under tension, such as, for example, rollers, curlers and the like.

The hair can also be shaped without the aid of external means, simply with the fingers.

Before carrying out the following optional rinsing step (ii), the hair onto which the reducing composition has been applied should, conventionally, be left to stand for a few minutes, generally from 5 minutes to one hour, such as from 10 to 30 minutes, so as to give the reducing agent enough time to act correctly on the hair.

In the optional second step of the process (step (ii)), the hair impregnated with the reducing composition is then rinsed thoroughly with an aqueous composition.

Next, in a third step (step (iii)), the oxidizing composition of the invention is applied to the hair thus rinsed, with the aim of fixing the new shape given to the hair.

As in the case of the application of the reducing composition, the hair onto which the oxidizing composition has been applied is then, conventionally, left for a standing or waiting phase lasting a few minutes, generally, for example, from 3 to 30 minutes, and as a further example, from 5 to 15 minutes.

If the hair was maintained under tension by external means, these means (rollers, curlers or the like) can be removed from the hair before or after the fixing step.

Lastly, in the final step of the process according to the invention (step (iv)), which is also optional, the hair impregnated with the oxidizing composition is rinsed thoroughly, generally with water.

Hair which is soft and easy to disentangle can finally be obtained. The hair can also be wavy.

The oxidizing composition according to the invention can also be used in a process for bleaching keratin fibers, such as human hair.

The bleaching process according to the invention comprises a step of applying an oxidizing composition according to the invention to the keratin fibers in the presence or absence of an auxiliary oxidizing agent. Conventionally, a second step of the bleaching process according to the invention is a step of rinsing the keratin fibers.

The medium which is suitable for the keratin fibers (or the support) for the ready-to-use dye compositions and for the oxidizing compositions used for the permanent reshaping or bleaching of keratin fibers in accordance with the invention generally comprises water or a mixture of water and at least one organic solvent to dissolve the compounds which would not be sufficiently soluble in water. By way of organic solvent, mention may be made, for example, of C₁-C₄ alkanols such as ethanol and isopropanol; polyols and their ethers such as glycerol, 2-butoxyethanol, propylene glycol, propylene glycol monomethyl ether, diethylene glycol monoethyl ether and monomethyl ether, and aromatic alcohols such as benzyl alcohol or phenoxyethanol, similar products and mixtures thereof.

The solvents can be present, for example, in proportions such as from 1% to 40% by weight approximately relative to the total weight of the dye composition, and as a further example, from 5% to 30% by weight approximately.

The pH of the ready-to-use dye compositions and of the oxidizing compositions used for the permanent reshaping or bleaching of the keratin fibers in accordance with the invention is chosen such that the enzymatic activity of the at least one enzyme chosen from 2-electron and 4-electron oxidoreductases and peroxidases is not adversely affected. For example, the pH generally ranges from 5 to 11 approximately, and, as a further example, from 6.5 to 10 approximately. It can be adjusted to the desired value using acidifying or basifying agents usually used for dyeing keratin fibers.

Among the acidifying agents, mention may be made, by way of example, of inorganic or organic acids such as hydrochloric acid, orthophosphoric acid, sulphuric acid, carboxylic acids such as acetic acid, tartaric acid, citric acid or lactic acid, and sulphonic acids.

Among the basifying agents, mention may be made, by way of example, of aqueous ammonia, alkaline carbonates, alkanolamines such as mono-, di- and triethanolamines, 2-methyl-2-aminopropanol and derivatives thereof, sodium hydroxide, potassium hydroxide and the compounds of formula (IV) below:

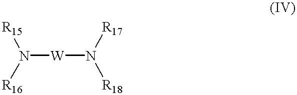

in which W is a propylene residue optionally substituted with a hydroxyl group or a C₁-C₄ alkyl radical; R₁₅, R₁₆, R₁₇ and R₁₈, which may be identical or different, represent a hydrogen atom or a C₁-C₄ alkyl or C₁-C₄ hydroxyalkyl radical.

The ready-to-use dye compositions and the oxidizing compositions for the permanent reshaping or bleaching of keratin fibers in accordance with the invention can also comprise various adjuvants used conventionally in compositions for dyeing, permanently reshaping or bleaching the hair, such as anionic, nonionic, amphoteric or zwitterionic surfactants or mixtures thereof, anionic polymers other than those of the invention, nonionic, amphoteric or zwitterionic polymers or mixtures thereof, inorganic or organic thickeners, antioxidants, penetration agents, sequestering agents, fragrances, buffers, dispersing agents, conditioners such as, for example, silicones, film-forming agents, preserving agents and opacifiers.

Needless to say, a person skilled in the art will take care to select this or these optional complementary compound(s) such that the advantageous properties intrinsically associated with the compositions in accordance with the invention are not, or are not substantially, adversely affected by the addition or additions envisaged.

The ready-to-use dye compositions and the oxidizing compositions used for the permanent reshaping or bleaching of keratin fibers in accordance with the invention can be in various forms, such as in the form of liquids, creams or gels, which are optionally pressurized, or in any other form which is suitable for dyeing, permanently reshaping or bleaching keratin fibers, such as human hair.

In the case of a ready-to-use dye composition, the oxidation dyes(s) and the at least one enzyme chosen from 2-electron and 4-electron oxidoreductases and peroxidases are present in the said composition, which must be free of oxygen gas, so as to avoid any premature oxidation of the oxidation dye(s) during storage of the said composition.

The examples which follow illustrate the invention without being limiting in nature.

### EXAMPLE 1

The conservation of the enzymatic activity of uricase, on the one hand, in a medium containing a thickener of the prior art, Aculyn 22, and, on the other hand, in the medium according to the present invention, i.e. with Stabileze QM, was studied.

A composition B according to the invention was thus compared with a composition A according to the prior art (amounts expressed in grams of Active Material).

The amounts of Aculyn 22 and of Stabileze QM were adjusted to obtain an identical and cosmetically acceptable thickening of the final compositions.

| | B | A |
|---|---|---|
| | Composition | Composition |
| | according to | according to |
| | the invention | the prior art |
| Stabileze QM (I.S.F.) | 1 | |
| Aculyn 22 (Rohm & Haas) | | 2.5 |
| Polyglyceryl monooleate (10 mol) | 1 | 1 |
| N-Acetyl-L-cysteine | 0.1 | 0.1 |
| 2-Amino-2-methyl-1-propanol qs pH | 9.5 | 9.5 |
| Uricase | 20,000 U | 20,000 U |
| Uric acid | 1 | 1 |
| Demineralized water qs | 100 | 100 |

The uricase activity in the oxidizing support is monitored by oximetry using a Clark electrode which records the decrease in the concentration of dissolved oxygen during the oxidation of uric acid to allantoin by uricase:

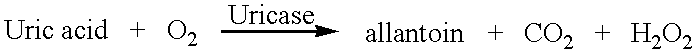

The rate of consumption of the dissolved oxygen in the support is compared with that obtained in standard solutions of uricase, which makes it possible to deduce the enzymatic activity in the medium studied.

### Results

3 assays were performed for each measurement of activity.

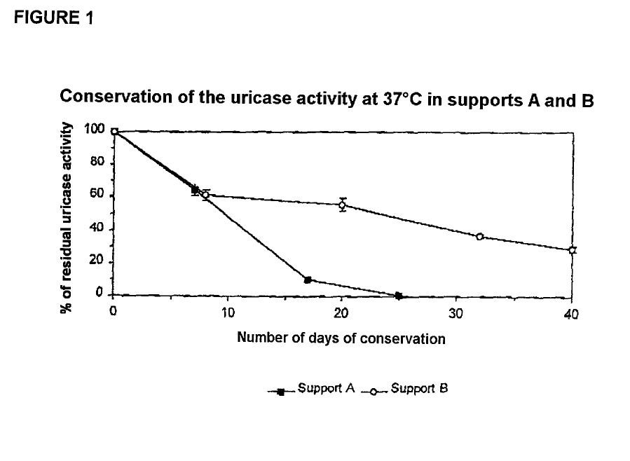

The results have been given in terms of a mean ±5% confidence interval in FIG. 1, which shows the percent of residual uricase activity as a function of time in supports A and B.

The results in FIG. 1 show that the composition comprising Stabileze QM is significantly superior to the prior art, in terms of conservation of the uricase activity.

### EXAMPLES 2, 3 AND 4

The ready-to-use dye compositions below were prepared: (amounts expressed in grams)

| Compositions | 2 | 3 | 4 |
|---|---|---|---|
| Stabileze QM (I.S.F.) | 1 | 1 | 1 |
| para-Phenylenediamine | 0.324 | 0.324 | 0.324 |
| 2-Methyl-5-aminophenol | 0.369 | | |
| 2,4-Diamino-1-(β- | | 0.723 | |
| hydroxyethyloxy)benzene | | | |
| dihydrochloride | | | |
| 1-Amino-2-methoxy-4,5- | | | 0.6108 |
| methylenedioxybenzene | | | |
| Polyglyceryl monooleate | 1 | 1 | 1 |
| (10 mol) | | | |
| N-Acetyl-L-cysteine | 0.1 | 0.1 | 0.1 |
| 2-Amino-2-methyl-1-propanol | 9.5 | 9.5 | 9.5 |
| qs pH | | | |
| Uricase | 1 | 1 | 1 |
| | i.e. 20,000 U | i.e. 20,000 U | i.e. 20,000 U |
| Uric acid | 1 | 1 | 1 |
| Demineralized water qs | 100 | 100 | 100 |

In parallel, compositions 5, 6 and 7 of the prior art, which differ from compositions 2, 3 and 4 above only by the presence of 2.5% of Aculyn 22 instead of 1% Stabileze, were prepared.

Each ready-to-use dye composition 2, 3, 4, 5, 6 and 7 was applied to locks of natural grey hair containing 90% white hairs and to locks of permanent-waved grey hair containing 90% white hairs, for 30 minutes at room temperature. The hair was then rinsed, washed with a standard shampoo and then dried.

Results: the locks of hair obtained with compositions 2, 3 and 4 were dyed uniformly in relatively unselective shades that are as intense and as chromatic as the locks of hair for the prior-art compositions 5, 6 and 7.

What is claimed is:

1. A cosmetic oxidizing composition for treating keratin fibers comprising, in a medium suitable for treating the keratin fibers:
(a) at least one enzymatic oxidizing system comprising at least one enzyme chosen from 2-electron and 4-electron oxidoreductases and peroxidases;
(b) at least one maleic anhydride/(C₁-C₅)alkyl vinyl ether crosslinked copolymer; and
(c) optionally a donor for said at least one enzyme.

2. The composition of claim 1, wherein said keratin fibers are human keratin fibers.

3. The composition of claim 1, wherein said keratin fibers are human hair.

4. The composition of claim 1, wherein said 2-electron oxidoreductases are chosen from pyranose oxidases, glucose oxidases, glycerol oxidases, lactate oxidases, pyruvate oxidases, uricases, choline oxidases, sarcosine oxidases, bilirubin oxidases, and amino acid oxidases; and
a donor for the at least one 2-electron oxidoreductases enzyme.

5. The composition of claim 1, said 2-electron oxidoreductases are chosen from uricases of animal, microbiological, and biotechnological origins.

6. The composition of claim 1, wherein the donor is chosen from uric acid and salts thereof.

7. The composition of claim 1, wherein said 4-electron oxidoreductases are chosen from laccases, tyrosinases, catechol oxidases, and polyphenol oxidases.

8. The composition of claim 7, wherein said 4-electron oxidoreductases are chosen from laccases of plant, animal, fungal, and bacterial origins, and from laccases obtained by biotechnology.

9. The composition of claim 8, wherein said laccases are of plant origin and are chosen from laccases present in extracts of Anacardiacea plants, Podocarpacea plants, Rosmarinus off., Solanum tuberosum, Iris sp., Coffea sp., Daucus carrota, Vinca minor, Persea americana, Catharanthus roseus, Musa sp., Malus pumila, Gingko biloba, Monotropa hypopithys (Indian pipe), Aesculus sp., Acer pseudoplatanus, Prunus persica, and Pistacia palaestina.

10. The composition of claim 8, wherein said laccases are chosen from laccases of fungal origin and obtained by biotechnology.

11. The composition of claim 10, wherein said laccases are chosen from the laccases obtained from Polyporus versicolor, Rhizoctonia praticola, Rhus vernicifera, Scytalidium, Polyporus pinsitus, Myceliophthora thermophila, Rhizoctonia solani, Pyricularia orizae, Trametes versicolor, Fomes fomentarius, Chaetomium thermophile, Neurospora crassa, Colorius versicol, Botrytis cinerea, Rigidoporus lignosus, Phellinus noxius, Pleurotus ostreatus, Aspergillus nidulans, Podospora anserina, Agaricus bisporus, Ganoderma lucidum, Glomerella cingulata, Lactarius piperatus, Russula delica, Heterobasidion annosum, Thelephora terrestris, Cladosporium cladosporioides, Cerrena unicolor, Coriolus hirsutus, Ceriporiopsis subvermispora, Coprinus cinereus, Panaeolus papilionaceus, Panaeolus sphinctrinus, Schizophyllum commune, Dichomitius squalens, and variants thereof.

12. The composition of claim 1, wherein said peroxidases are chosen from simplex peroxidases.

13. The composition of claim 1, said peroxidases are chosen from catalases.

14. The composition of claim 1, wherein said peroxidases are chosen from NADH peroxidases, fatty acid peroxidases, NADPH peroxidases, cytochrome-c peroxidases, iodide peroxidases, chloride peroxidases, L. ascorbate peroxidases, and glutathione peroxidases; and
wherein said composition further includes a donor for said peroxidases.

15. The composition of claim 1, wherein said peroxidases are chosen from peroxidases of animal, plant, fungal, and bacterial origin, and those obtained by biotechnology.

16. The composition of claim 15, wherein said peroxidases are chosen from peroxidases obtained from apple, apricot, barley, black radish, beetroot, cabbage, carrot, corn, cotton, garlic, grape, mint, rhubarb, soybean, spinach, inky cap, cow's milk, and at least one microorganism.

17. The composition of claim 16, wherein the at least one microorganism is chosen from Acetobacter peroxidans, Staphylococcus faecalis, and Arthromyces ramosus.

18. The composition of claim 1, wherein the composition comprises at least one oxidoreductase in an amount ranging from 0.01% to 20% by weight relative to the total weight of the composition.

19. The composition of claim 18, wherein the composition comprises the at least one oxidoreductase in an amount ranging from 0.1% to 5% by weight relative to the total weight of the composition.

20. The composition of claim 1, wherein the composition comprises at least one oxidoreductase in an amount ranging from approximately 10 to 10⁸ units U per 100 grams of the composition.

21. The composition of claim 1, wherein said at least one enzyme is chosen from 4-electron oxidoreductases chosen from laccases in an amount ranging from approximately 10,000 to 40×10⁶ U units per 100 grams of the composition.

22. The composition of claim 1, wherein said at least one enzyme is chosen from peroxidases in an amount ranging from 0.0001% to 20% by weight relative to the total weight of the composition.

23. The composition of claim 22, wherein said amount ranges from 0.001% to 10% by weight relative to the total weight of the composition.

24. The composition of claim 1, wherein said at least one enzyme is chosen from peroxidases chosen from simplex peroxidases in an amount ranging from approximately 25 to 5×10⁶ activity units per 100 grams of composition.

25. The composition of claim 1, wherein the maleic anhydride/(C₁-C₅)alkyl vinyl ether crosslinked copolymer, when hydrolysed and neutralized, and dissolved in water at a concentration ranging from 0.5% to 1%, at a pH ranging from 5 to 10, yields a solution having a viscosity, measured at 25° C. using a Brookfield RTV viscometer with a No. 7 rotor at 20 rpm, ranging from 45,000 to 70,000 mPa.s.

26. The composition of claim 1, wherein the maleic anhydride/(C₁-C₅)alkyl vinyl ether crosslinked copolymer is formed from a process that produces the copolymer in the form of particles having a size of less than 850 microns.

27. The composition of claim 26, wherein the particle size is less than 75 microns.

28. The composition of claim 27, wherein the particle size ranges from 1 to 15 microns.

29. The composition of claim 1, wherein the maleic anhydride/(C₁-C₅)alkyl vinyl ether crosslinked copolymer is a maleic anhydride/methyl vinyl ether copolymer crosslinked with at least one crosslinker chosen from divinyl ethers of aliphatic diols and divinyl ethers of polyethylene glycol.

30. The composition of claim 1, wherein the maleic anhydride/(C₁-C₅)alkyl vinyl ether crosslinked copolymer is a maleic anhydride/methyl vinyl ether copolymer crosslinked with at least one crosslinker chosen from 1,7-octadiene, 1,9-decadiene, divinylbenzene, N,N′-bis(methyleneacrylamide), polyethylene glycol diacrylate, propylene glycol diacrylate, trimethylolpropane triacrylate, and polyhydric alcohols esterified with acrylic acid.

31. The composition of claim 1, wherein the maleic anhydride/(C₁-C₅)alkyl vinyl ether crosslinked copolymer is a maleic anhydride/methyl vinyl ether copolymer crosslinked with 1,9-decadiene.

32. The composition of claim 1, wherein the composition comprises the maleic anhydride/(C₁-C₅)alkyl vinyl ether crosslinked copolymer in an amount ranging from 0.001% to 10% by weight relative to the total weight of the composition.

33. The composition of claim 32, wherein the composition comprises the maleic anhydride/(C₁-C₅)alkyl vinyl ether crosslinked copolymer in an amount ranging from 0.01% to 5% by weight relative to the total weight of the composition.

34. The composition of claim 1, wherein the medium suitable for treating the keratin fibers is chosen from water and a mixture of water and at least one organic solvent.

35. The composition of claim 34, wherein the medium comprises the at least one organic solvent in an amount ranging from 1% to 40% by weight relative to the total weight of the composition.

36. The composition of claim 34, wherein the medium comprises the at least one organic solvent in an amount ranging from 5% to 30% by weight relative to the total weight of the composition.

37. The composition of claim 1, wherein the composition has a pH ranging from 5 to 11.

38. The composition of claim 1, wherein the composition has a pH ranging from 6.5 to 10.

39. The composition of claim 1, further comprising at least one cosmetic adjuvant chosen from anionic, nonionic, amphoteric, and zwitterionic surfactants; anionic polymers different from the at least one maleic anhydride/(C₁-C₅)alkyl vinyl ether cross-linked copolymer, nonionic, amphoteric, and zwitterionic polymers, inorganic and organic thickeners; antioxidants; penetration agents; sequestering agents; fragrances; buffers; dispersing agents; conditioners; film-forming agents; preserving agents; and opacifiers.

40. A ready-to-use composition for oxidation dyeing of keratin fibers, comprising, in a medium suitable for the oxidation dyeing the keratin fibers:
(a) at least one enzymatic oxidizing system comprising at least one enzyme chosen from 2-electron and 4-electron oxidoreductases and peroxidases;
(b) at least one maleic anhydride/(C₁-C₅)alkyl vinyl ether crosslinked copolymer;
(c) optionally a donor for said at least one enzyme; and
(d) at least one oxidation dye.

41. The composition of claim 40, wherein the keratin fibers are human keratin fibers.

42. The composition of claim 40, wherein the keratin fibers are human hair.

43. The composition of claim 40, wherein the at least one oxidation dye comprises at least one oxidation base chosen from ortho- and para-phenylenediamines, double bases, ortho- and para-aminophenols, and heterocyclic bases, and acid addition salts thereof.

44. The composition of claim 43, wherein the composition comprises the at least one oxidation bases in an amount ranging from 0.0005% to 12% by weight relative to the total weight of the composition.

45. The composition of claim 40, wherein the at least one oxidation dye comprises at least one coupler chosen from meta-phenylenediamines, meta-aminophenols, meta-diphenols, mono- and polyhydroxylated naphthalene derivatives, sesamol and derivatives thereof, heterocyclic couplers, and acid addition salts thereof.

46. The composition of claim 45, wherein the composition comprises the at least one coupler in an amount ranging from 0.0001% to 10% by weight relative to the total weight of the composition.

47. The composition of claim 43, wherein the at least one oxidation base is an acid addition salt with an acid chosen from the hydrochlorides, hydrobromides, sulphates, tartrates, lactates, and acetates.

48. The composition of claim 45, wherein the at least one coupler is an acid addition salt with an acid chosen from the hydrochlorides, hydrobromides, sulphates, tartrates, lactates, and acetates.

49. The composition of claim 40, further comprising at least one direct dye in an amount ranging from 0.001% to 20% by weight relative to the total weight of the composition.

50. A process for dyeing keratin fibers, comprising:
applying a composition to the keratin fibers for a period time sufficient to develop a desired color,
wherein said composition is a ready-to-use composition for oxidation dyeing of the keratin fibers, comprising, in a medium suitable for oxidation dyeing the keratin fibers:
(a) at least one enzymatic oxidizing system comprising at least one enzyme chosen from 2-electron and 4-electron oxidoreductases and peroxidases;
(b) at least one maleic anhydride/(C₁-C₅)alkyl vinyl ether crosslinked copolymer;
(c) optionally a donor for said at least one enzyme; and
d) at least one oxidation dye.

51. The process of claim 50, wherein the keratin fibers are human keratin fibers

52. The process of claim 50, wherein the keratin fibers are human hair.

53. The process according to claim 50, further comprising:
prior to applying, separately storing a composition (A) comprising, in a medium suitable for dyeing the keratin fibers, the at least one oxidation dye, and a composition (B) comprising, in a medium suitable for dyeing the keratin fibers, the at least one enzyme and the optional donor; and
subsequently mixing the separately stored compositions together at a time of use, before applying the mixture to the keratin fibers,
wherein at least one of the compositions (A) and (B) further comprises the maleic anhydride/(C₁-C₅)alkyl vinyl ether crosslinked copolymer.

54. The process of claim 53, wherein composition (B) further comprises at least one donor for the at least one enzyme.

55. A multi-compartment dyeing device or kit, comprising:
a first compartment comprising composition (A) comprising, in a medium suitable for dyeing the keratin fibers, at least one oxidation dye; and
a second compartment comprising composition (B) comprising, in a medium suitable for dyeing the keratin fibers, at least one enzyme chosen from 2-electron and 4-electron oxidoreductases and peroxidases and optionally, a donor for said at least one enzyme,
wherein at least one of the compositions (A) and (B) further comprises at least one maleic anhydride/(C₁-C₅)alkyl vinyl ether crosslinked copolymer.

56. The multi-compartment dyeing device or kit of claim 55, wherein composition (B) further comprises at least one donor for the at least one enzyme

57. A process for treating keratin fibers in order to obtain a permanent reshaping of these fibers, in the form of permanent-waved hair, comprising:
(i) applying to the keratin fibers a reducing composition;
(ii) placing the keratin fibers under mechanical tension at least one of before, during, and after applying the reducing composition; and
(iii) applying to the keratin fibers an oxidizing composition comprising, in a medium suitable for treating the keratin fibers:
(a) at least one enzymatic oxidizing system comprising at least one enzyme chosen from 2-electron and 4-electron oxidoreductases and peroxidases;
(b) at least one maleic anhydride/(C₁-C₅)alkyl vinyl ether crosslinked copolymer; and
(c) optionally a donor for said at least one enzyme.

58. The process of claim 57, further comprising rinsing the keratin fibers at least one of before and after applying the oxidizing composition.

59. The process of claim 57, wherein the keratin fibers are human hair.

60. A process for bleaching keratin fibers, comprising
applying to the keratin fibers an oxidizing composition comprising:
(a) at least one enzymatic oxidizing system comprising at least one enzyme chosen from 2-electron and 4-electron oxidoreductases and peroxidase;
(b) at least one maleic anhydride/(C₁-C₅)alkyl vinyl ether crosslinked copolymer; and
(c) optionally a donor for said at least one enzyme; and
subsequently rinsing the keratin fibers.

61. The process of claim 60, wherein the oxidizing composition further comprises an additional oxidizing agent.

62. The process of claim 60, wherein the keratin fibers are human hair.

63. A process for improving the conservation of the enzymatic activity of an oxidizing composition, comprising including an effective amount of a maleic anhydride/(C₁-C₅)alkyl vinyl ether crosslinked copolymer in the oxidizing composition;
wherein the oxidizing composition is an oxidizing composition for at least one of dyeing, permanently reshaping, and bleaching keratin fibers, and comprises, as oxidizing system, at least one enzyme chosen from 2-electron and 4-electron oxidoreductases and peroxidases and optionally, a donor for said at least one enzyme.

64. The process of claim 63, wherein the keratin fibers are human hair.

65. The process of claim 63, wherein the oxidizing system comprises at least one uricase and uric acid, and the effective amount of a maleic anhydride/methyl vinyl ether copolymer, crosslinked with 1,9-decadiene, is included in the oxidizing composition.
